(19) United States
(12) Patent Application Publication
(10) Pub. No.: US 2026/0085029 A1
(43) Pub. Date: Mar. 26, 2026

(54) METHOD FOR PRODUCING BIOBASED ALPHA-BETA-UNSATURATED CARBOXYLIC ACIDS FROM POLY(3-HYDROXYALKANOATE) CONTAINED IN BIOMASS

(71) Applicant: Arkema France, Colombes (FR)

(72) Inventors: Yves CABON, Saint Avold Cedex (FR); Akan ARAYICI, Saint Avold Cedex (FR); Jean-Jacques GESLOT, Saint Avold Cedex (FR)

(21) Appl. No.: 19/108,478

(22) PCT Filed: Sep. 4, 2023
(86) PCT No.: PCT/EP2023/074177

§ 371 Date: Mar. 4, 2025

(30) Foreign Application Priority Data
Sep. 6, 2022 (FR) FR22.08916

(51) Int. Cl. C07C 51/09; C07C 51/50

(52) U.S. Cl. CPC C07C 51/09; C07C 51/50

(57) ABSTRACT

The present invention relates to a method for producing biobased α-β-unsaturated carboxylic acids from poly(3-hydroxyalkanoate) (P3HA) contained in biomass by extraction of the P3HA using a solvent, followed by separation of insoluble organic waste and then thermolysis of the P3HA-solvent mixture in the presence of the solvent. The solvent is selected such that it provides good dissolution of the P3HA and does not boil under the thermolysis conditions.

### TECHNICAL FIELD

The present invention relates to a process for producing biobased α,β-unsaturated carboxylic acids by extracting a poly(3-hydroxyalkanoate) from biomass using a solvent, followed by thermolysis of said polymer in the presence of said solvent.

### PRIOR ART AND TECHNICAL PROBLEM

α,β-Unsaturated carboxylic acids are nowadays produced industrially mainly from feedstocks of fossil origin. For example, acrylic acid is obtained by oxidation of propylene, or methacrylic acid can be obtained by oxidation of isobutylene.

One possible way of obtaining these α,β-unsaturated carboxylic acids is the thermolysis at temperatures of 150 to 300° C. of the corresponding poly(3-hydroxyalkanoate) (P3HA), according to the following reaction:

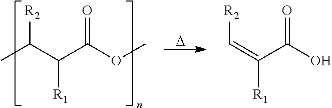

R₁=H or alkyl and R₂=H or alkyl; n is a number greater than 30

If R₁=R₂=H:
Poly(3-hydroxyalkanoate)=poly(3-hydroxypropionate) (P3HP);
α,β-Unsaturated carboxylic acid=propenoic acid (acrylic acid).

If R₁=methyl and R₂=H:
Poly(3-hydroxyalkanoate)=poly(3-hydroxyisobutyrate) (P3HIB);
α,β-Unsaturated carboxylic acid=isobutenoic acid (methacrylic acid).

If R₁=H and R₂=methyl:
Poly(3-hydroxyalkanoate)=poly(3-hydroxybutyrate) (P3HB);
α,β-Unsaturated carboxylic acid=but-2-enoic acid (crotonic acid).

If R1=Hand R₂=ethyl:
Poly(3-hydroxyalkanoate) is poly(3-hydroxyvalerate) (P3HV);
α,β-Unsaturated carboxylic=pent-2-enoic acid

These poly(3-hydroxyalkanoates) can themselves be obtained beforehand by chemical transformations of feedstocks of fossil origin, but also by fermentation of biomass.

There is strong market demand for these α,β-unsaturated carboxylic acids, which are used as monomers in numerous applications, to be obtained from biobased feedstocks. These biobased feedstocks are derived from renewable organic matter (biomass) of biological origin (microorganisms, plants or animals).

A potential problem with such a process is that the P3HA obtained by fermentation is present inside the cell. The thermolysis is therefore carried out in the presence of the cell membrane, which presents problems with clogging of the reactor or with the presence of impurities in the end product, or else necessitates a preliminary P3HA extraction step, which can be complex and costly.

A number of solutions to this problem have been proposed.

Document U.S. Pat. No. 9,850,192 describes a process for producing acrylic acid from a genetically modified microbial biomass metabolizing glucose or any other renewable feedstock to produce a poly-3-hydroxypropionate (P3HP) homopolymer or copolymer inside microbial cells. Said process comprises a step of thermolysis of the washed/dried/milled P3HP-containing biomass in the presence of a catalyst. This process is able to produce acrylic acid effectively, but there is a risk that the residue present in the reactor after thermolysis will be pasty and sticky, which could complicate its transfer to industrial scale.

Another solution is to first extract the P3HA from the biomass using an organic solvent before subjecting it to thermolysis. Document US 20150376152 describes in example 6 the extraction of P3HP from biomass using an organic solvent such as 2-butanone. The solvent is then removed by distillation before performing the thermolysis of P3HP to acrylic acid.

The inventors have now surprisingly discovered that it is possible to simplify the procedure for the extraction from biomass and subsequent thermolysis of P3HA without performing an additional step of separating the solvent used for the extraction of P3HA.

The invention accordingly proposes to provide a simple and easily implementable solution for reducing clogging phenomena and the presence of impurities in the end product and thus maintain high reliability and increased productivity in processes for producing α,β-unsaturated carboxylic acids from poly(3-hydroxyalkanoates) obtained by fermentation.

### SUMMARY OF THE INVENTION

The proposed technical solution is to use for the extraction of P3HA from biomass an organic solvent in which P3HA is highly soluble but that also has a boiling point sufficiently high that the thermolysis step can be carried out directly on this P3HA-solvent mixture without having to first remove the extraction solvent.

The present invention provides a process for producing a biobased α,β-unsaturated carboxylic acid, said process comprising the following steps:
mixing a biomass comprising poly(3-hydroxyalkanoate) with a solvent capable of solubilizing P3HA,
separating the organic waste insoluble in said solvent, comprising cell membranes, from the P3HA-solvent mixture,
subjecting said PHA-solvent mixture to a thermolysis step resulting in, firstly, said α,β-unsaturated carboxylic acid and, secondly, said solvent.

According to various implementations, said process comprises the following features, where appropriate in combination. Stated contents are expressed in weight, unless otherwise stated. In stated ranges of values, the limits are included.

According to one embodiment, the poly(3-hydroxyalkanoate) used in the process comprises a single type of 3-hydroxyalkanoate units and the product formed is therefore composed of a single α,β-unsaturated carboxylic acid.

According to one embodiment, the poly(3-hydroxyalkanoate) contains the 3-hydroxypropionate unit and at least one of the α,β-unsaturated carboxylic acids produced is acrylic acid.

According to one embodiment, the poly(3-hydroxyalkanoate) is poly(3-hydroxypropionate) and the α,β-unsaturated carboxylic acid produced is acrylic acid.

According to one embodiment, the poly(3-hydroxyalkanoate) contains the 3-hydroxybutyrate unit and at least one of the α,β-unsaturated carboxylic acids produced is crotonic acid.

According to one embodiment, the poly(3-hydroxyalkanoate) is poly(3-hydroxybutyrate) and the α,β-unsaturated carboxylic acid produced is crotonic acid.

According to one embodiment, the poly(3-hydroxyalkanoate) contains the 3-hydroxyisobutyrate unit and at least one of the α,β-unsaturated carboxylic acids produced is methacrylic acid.

According to one embodiment, the poly(3-hydroxyalkanoate) is poly(3-hydroxyisobutyrate) and the α,β-unsaturated carboxylic acid produced is methacrylic acid.

According to one embodiment, the poly(3-hydroxyalkanoate) used in the process comprises a plurality of different 3-hydroxyalkanoate units and the product formed is therefore composed of a mixture of different α,β-unsaturated carboxylic acids. Examples of P3HA copolymers are poly-3-hydroxybutyrate-co-3-hydroxypropionate, poly-3-hydroxybutyrate-co-3-hydroxyvalerate (poly-3-HB-co-3HV).

According to one embodiment, the biomass host is a bacterium, yeast, fungus, alga, cyanobacterium or a mixture of two or more of these elements.

According to one embodiment, the biomass used is pretreated by means of washing, drying and milling operations to yield a biomass containing at least 50% by weight of P3HA.

According to one embodiment, the step of extracting the P3HA from the biomass with a solvent comprises a separation of organic waste insoluble in said solvent, for example the cell membranes, from the P3HA-solvent mixture, which is achieved by filtration or by centrifugation.

According to one embodiment, the step of extracting P3HA from the biomass with a solvent takes place at a temperature of 20 to 130° C.

According to one embodiment, the step of extracting P3HA from the biomass with a solvent takes place batchwise.

According to one embodiment, the step of extracting P3HA from the biomass with a solvent takes place continuously.

According to one embodiment, the thermolysis reaction of P3HA in solution in the solvent takes place at a temperature of from 130 to 300° C. and at a pressure of from 1 to 101 kPa (atmospheric pressure).

According to one embodiment, the solvent used to extract P3HA present in the biomass has a boiling point such that it does not boil under the temperature and pressure conditions of the thermolysis reaction.

According to one embodiment, the thermolysis reaction takes place in the presence of one or more polymerization inhibitors.

The polymerization inhibitors used in the process according to the invention are selected from inhibitors conventionally used in existing industrial processes for the production of α,β-unsaturated carboxylic acids. These include phenol derivatives such as hydroquinone (HQ) and derivatives thereof such as hydroquinone methyl ether (HQME), 2,6-di-tert-butyl-4-methylphenol (BHT) or 2,4-dimethyl-6-tert-butylphenol (Topanol A); phenothiazine and derivatives thereof; nitroxide compounds such as 4-hydroxy-2,2,6,6-tetramethylpiperidin-1-oxyl (4-OH-TEMPO); and amino compounds such as para-phenylenediamine derivatives.

According to a preferred embodiment, at least one of said polymerization inhibitors is hydroquinone methyl ether (HQME).

According to one embodiment, the thermolysis reaction of the poly(3-hydroxyalkanoate) takes place batchwise.

According to one embodiment, the thermolysis reaction of the poly(3-hydroxyalkanoate) takes place continuously.

According to one embodiment, the process according to the invention comprises a step of condensing the vapor of the α,β-unsaturated carboxylic acid(s) obtained by the thermolysis reaction of poly(3-hydroxyalkanoate), followed by one or more purification steps. The purification operations can generally include distillations, liquid/liquid extractions, separations using a film evaporator, or crystallizations.

According to one embodiment, the process according to the invention comprises a step of recycling the solvent used for extracting the P3HA from the biomass, at the end of the thermolysis step. It is advantageous when the solvent can be recycled to the extraction step without having to be distilled.

The present invention meets the need expressed in the prior art. It makes it possible to avert the risk of the thermolysis reactor becoming clogged and/or of the α,β-unsaturated carboxylic acids end product containing impurities originating from cell membranes, without performing a step of separating the solvent used for the extraction of P3HA.

This solution has two advantages: it saves having to distill off the solvent, which is energy-intensive, and the thermolysis of P3HA is carried out not on the bulk, but on the solution, which reduces the risk of the thermolysis reactor becoming clogged.

The invention will now be described in more detail in the description that follows.

### DETAILED DESCRIPTION OF THE INVENTION

The aim of the invention is to produce biobased α,β-unsaturated carboxylic acids on an industrial scale by thermolysis of poly(3-hydroxyalkanoate) contained in the biomass, while limiting problems with clogging of the thermolysis reactor and/or with the presence of impurities in the end product.

The invention proposes to provide a process that makes it possible to reduce or eliminate this risk of clogging.

The invention is based on the use for the extraction of P3HA from biomass of an organic solvent in which P3HA is highly soluble but that also has a boiling point sufficiently high that the thermolysis step can be carried out directly on this P3HA-solvent mixture without having to remove the extraction solvent first.

The term “biomass” means organic matter originating from plants (including microalgae), animals, bacteria or fungi that is employable as a source of biobased feedstocks, as opposed to feedstocks of fossil origin.

In the process according to the invention, the first step uses genetically modified host biomass derived from genetic engineering. According to one embodiment, the biomass host is a bacterium, yeast, fungus, alga, cyanobacterium or a mixture of two or more of these elements.

The biomass is obtained by a prior step of culturing a recombinant host with a renewable feedstock. According to one embodiment, the renewable feedstock is selected from glucose, fructose, sucrose, arabinose, maltose, lactose, xylose, ethanol, methanol, glycerol, fatty acids, vegetable oils and biomass-derived synthesis gas or a combination thereof.

According to one embodiment, the biomass used in the process according to the invention comes from a process of bacterial fermentation of sugars or lipids.

Depending on the culture conditions and on the variety of microorganism used, poly(3-hydroxyalkanoate) (P3HA) homo- or copolymers with different 3-hydroxyalkanoic acids are formed.

According to one embodiment, the biomass used is pretreated by means of washing, drying and milling operations to yield a biomass containing at least 50% by weight of P3HA.

According to one embodiment, the solvent used to extract the P3HA present in the biomass is selected from polar organic solvents with a high boiling point, so that the solvent does not boil in the thermolysis step. Examples of such solvents are glycol diethers (glymes), such as tetraglyme, sulfur-containing sulfoxide or sulfone solvents, such as sulfolane or dimethyl sulfone, carbonate solvents, such as propylene carbonate, or phenol derivatives, such as pare-methoxyphenol (also termed hydroquinone methyl ether or HQME).

According to one embodiment, the extraction of P3HA present in the biomass takes place at a temperature of 20 to 130° C.

According to one embodiment of the invention, the solvent used in the process must be capable of solubilizing the P3HA in a content of greater than 5% by weight in the solution, preferably greater than 20%, at the temperature used during the extraction step.

According to one embodiment, the extraction is followed by a step of separating the P3HA-solvent mixture and the organic waste insoluble in said solvent, for example the cell membranes. Possible methods are filtration or centrifugation.

According to one embodiment of the invention, the steps of extracting P3HA from the biomass with a solvent and separating the P3HA-solvent mixture from the organic waste insoluble in said solvent can be carried out batchwise.

According to one embodiment of the invention, the steps of extracting P3HA from the biomass with a solvent and separating the P3HA-solvent mixture from the organic waste insoluble in said solvent can be carried out continuously.

The term “thermolysis” of poly(3-hydroxyalkanoate) (P3HA) means its chemical decomposition into α,β-unsaturated carboxylic acid that occurs under the effect of temperature. This term is synonymous with pyrolysis.

In the process according to the invention, the thermolysis reaction of the poly(3-hydroxyalkanoate)-solvent mixture takes place at a temperature of from 130 to 300° C., preferably from 170 to 230° C., and at a pressure of from 1 to 101 kPa (atmospheric pressure).

According to one embodiment of the invention, the step of thermolysis of the P3HA-solvent mixture can be carried out batchwise.

According to one embodiment of the invention, the step of thermolysis of the P3HA-solvent mixture can be carried out continuously.

The solvent recovered at the end of the thermolysis is advantageously recycled to the extraction step.

According to one embodiment of the invention, the solvent used in the process has a boiling point greater than 230° C. at atmospheric pressure.

According to one embodiment, the thermolysis of poly(3-hydroxyalkanoate) takes place in the absence of a catalyst. The use of catalysts makes it possible to accelerate the thermolysis kinetics and/or to reduce the thermolysis temperature. However, the use of a catalyst makes the process more complex and more difficult to implement on an industrial scale.

According to one embodiment, the reaction medium in the thermolysis reactor comprises at least one polymerization inhibitor, in a proportion of especially from 50 ppm to 5% by weight, in particular from 0.01% to 3% by weight, relative to the weight of the poly(3-hydroxyalkanoate).

The polymerization inhibitors are selected from inhibitors conventionally used in existing industrial processes for the production of α,β-unsaturated carboxylic acids. These include phenol derivatives such as hydroquinone (HQ) and derivatives thereof such as hydroquinone methyl ether (HQME), 2,6-di-tert-butyl-4-methylphenol (BHT) or 2,4-dimethyl-6-tert-butylphenol (Topanol A); phenothiazine and derivatives thereof; nitroxide compounds such as 4-hydroxy-2,2,6,6-tetramethylpiperidin-1-oxyl (4-OH-TEMPO); and amino compounds such as para-phenylenediamine derivatives.

According to one embodiment, said polymerization inhibitor is hydroquinone methyl ether (HQME).

According to a specific embodiment, the solvent used to extract the P3HA present in the biomass is hydroquinone methyl ether (HQME) used in molten form. After the step of separating the P3HA-HQME mixture from insoluble organic waste, the P3HA in solution in the molten HQME is then thermolyzed under conditions at which the HQME is partially volatile but does not boil, without supplementary addition of HQME. HQME thus at the same time acts not just as an extraction solvent and thermolysis solvent but also as a partially volatile polymerization inhibitor.

According to one embodiment, the temperature and pressure conditions in the thermolysis reactor are selected such that the α,β-unsaturated carboxylic acid or acids formed are in vapor form.

According to one embodiment, the invention relates to a process for producing biobased acrylic acid from P3HP contained in the biomass.

According to one embodiment, the invention relates to a process for producing biobased methacrylic acid from P3HiB contained in the biomass.

According to one embodiment, the invention relates to a process for producing biobased crotonic acid from P3HB contained in the biomass.

According to one embodiment, the invention relates to a process for producing a mixture of α,β-unsaturated carboxylic acids from a P3HA contained in the biomass, comprising a plurality of different 3-hydroxyalkanoate units.

According to one embodiment, the process according to the invention comprises a step of condensing the vapor of the α,β-unsaturated carboxylic acid(s) obtained by the thermolysis reaction of poly(3-hydroxyalkanoate), followed by one or more purification steps. Purification operations can generally include distillations, liquid/liquid extractions, separations using a film evaporator, or crystallizations.

According to a preferred embodiment, the P3HA contained in the biomass is P3HP, the extraction is performed using a solvent of sulfone type, such as sulfolane or dimethyl sulfone, and the thermolysis step of the P3HP-sulfone solvent mixture is carried out in the presence of hydroquinone methyl ether.

The examples that follow illustrate the present invention, but without limiting the scope thereof.

### EXPERIMENTAL SECTION

The examples are carried out on biomass containing 60% by weight of P3HP. The α,β-unsaturated carboxylic acid obtained after thermolysis is acrylic acid (AA). The step of extracting P3HP with a solvent is carried out by introducing 2 g of this biomass and 20 g of solvent into a glass test tube equipped with a magnetic stirrer bar. The medium is placed in an oil bath at 100° C. and stirred using a magnetic stirrer for 2 h. The insoluble organic waste is then separated from the P3HP-solvent mixture by centrifugation.

The step of thermolyzing P3HP in solution in the solvent is carried out by placing the medium obtained after the step of extraction and separation of the organic waste in a 50 mL two-necked flask; an addition of HQME inhibitor can be made. The flask is equipped on the side neck with a thermometer to monitor the temperature of the thermolysis medium and on the upper neck with a separation bridge leading to a water-cooled side condenser. The condenser leads to a receiver consisting of a 25 mL single-necked flask cooled by an ice bath. An air bleed allows the experiment to be performed under a partial vacuum.

Thermolysis of the Biomass in Solvent Medium without Extraction and Prior Separation of Organic Waste

### Experiment 1

2 g of biomass containing 60% of P3HP (i.e. 1.2 g of P3HP) and 20 g of sulfolane are introduced directly into the thermolysis assembly. 0.02 g of HQME is added as polymerization inhibitor. The thermolysis reaction is carried out at 200° C. and 20 kPa for 4 h. 1.1 g of AA having a purity greater than 90% is recovered in the overhead receiver. The residue in the thermolysis reactor is barely workable (liquid loaded with sticky solid).

### Experiment 2

2 g of biomass containing 60% of P3HP (i.e. 1.2 g of P3HP) and 20 g of tetraglyme are introduced directly into the thermolysis assembly. 0.02 g of HQME is added as polymerization inhibitor. The thermolysis reaction is carried out at 200° C. and 20 kPa for 4 h. 1.1 g of AA having a purity greater than 90% is recovered in the overhead receiver. The residue in the thermolysis reactor is barely workable (liquid loaded with sticky solid).

### Experiment 3

2 g of biomass containing 60% of P3HP (i.e. 1.2 g of P3HP) and 20 g of HQME are introduced directly into the thermolysis assembly. The thermolysis reaction is carried out at 200° C. and 20 kPa for 4 h. 0.9 g of AA having a purity greater than 90% is recovered in the overhead receiver. The residue in the thermolysis reactor is barely workable (liquid loaded with sticky solid).

### Experiment 4

2 g of biomass containing 60% of P3HP (i.e. 1.2 g of P3HP) and 20 g of dimethyl sulfone are introduced directly into the thermolysis assembly. The thermolysis reaction is carried out at 200° C. and 20 kPa for 4 h. 1.0 g of AA having a purity greater than 90% is recovered in the overhead receiver. The residue in the thermolysis reactor is barely workable (liquid loaded with sticky solid).

### Experiment 5

2 g of biomass containing 60% of P3HP (i.e. 1.2 g of P3HP) and 20 g of propylene carbonate are introduced directly into the thermolysis assembly. The thermolysis reaction is carried out at 200° C. and 70 kPa for 4 h. 0.8 g of AA having a purity greater than 90% is recovered in the overhead receiver. The residue in the thermolysis reactor is barely workable (liquid loaded with sticky solid).

Thermolysis after Prior Extraction of the Biomass with Methyl Isobutyl Ketone (MIBK) and Separation of Organic Waste

### Experiment 6

2 g of biomass containing 60% of P3HP (i.e. 1.2 g of P3HP) is extracted with 20 g of MIBK at 100° C. for 2 h.

The insoluble organic waste is separated by centrifugation and weighs 1.2 g. A maximum of 0.8 g of P3HP has therefore been extracted into the MIBK, i.e. two-thirds of the P3HP contained in the biomass.

The homogeneous P3HP-MIBK mixture is introduced directly into the thermolysis assembly. 0.02 g of HQME is added as polymerization inhibitor. The thermolysis reaction is carried out at 200° C. and 20 kPa for 4 h. 20.3 g of a homogeneous organic medium comprising 3% (i.e. 0.6 g) of AA dissolved in MIBK is recovered overhead. The residue in the thermolysis reactor is a sticky solid.

Thermolysis Aver Prior Extraction of the Biomass with a Solvent According to the Invention (Good Solvent for P3HP that does not Boil Under the Thermolysis Conditions)

### Experiment 7

2 g of biomass containing 60% of P3HP (i.e. 1.2 g of P3HP) is extracted with 20 g of sulfolane at 100° C. for 2 h.

The insoluble organic waste is separated by centrifugation and weighs 0.8 g. A maximum of 1.2 g of P3HP has therefore been extracted into the sulfolane, i.e. potentially all of the P3HP contained in the biomass.

The homogeneous P3HP-sulfolane mixture is introduced directly into the thermolysis assembly. 0.02 g of HQME is added as polymerization inhibitor. The thermolysis reaction is carried out at 200° C. and 20 kPa for 4 h. 1.1 g of AA having a purity greater than 90% is recovered in the overhead receiver. The residue in the thermolysis reactor is a clear liquid consisting essentially of sulfolane and which can be recycled to the extraction step.

### Experiment 8

2 g of biomass containing 60% of P3HP (i.e. 1.2 g of P3HP) is extracted with 20 g of tetraglyme at 100° C. for 2 h.

The insoluble organic waste is separated by centrifugation and weighs 0.8 g. A maximum of 1.2 g of P3HP has therefore been extracted into the tetraglyme, i.e. potentially all of the P3HP contained in the biomass.

The homogeneous P3HP-tetraglyme mixture is introduced directly into the thermolysis assembly. 0.02 g of HQME is added as polymerization inhibitor. The thermolysis reaction is carried out at 200° C. and 20 kPa for 4 h. 1.1 g of AA having a purity greater than 90% is recovered in the overhead receiver. The residue in the thermolysis reactor is a clear liquid consisting essentially of tetraglyme which can be recycled to the extraction step.

### Experiment 9

2 g of biomass containing 60% of P3HP (i.e. 1.2 g of P3HP) is extracted with 20 g of HQME at 100° C. for 2 h.

The insoluble organic waste is separated by centrifugation and weighs 0.8 g. A maximum of 1.2 g of P3HP has therefore been extracted into the HQME, i.e. potentially all of the P3HP contained in the biomass.

The homogeneous P3HP-EMI-IQ mixture is introduced directly into the thermolysis assembly. The thermolysis reaction is carried out at 200° C. and 20 kPa for 4 h. 0.9 g of AA having a purity greater than 90% is recovered in the overhead receiver. The residue in the thermolysis reactor is a clear liquid consisting essentially of HQME which can be recycled to the extraction step.

### Experiment 10

2 g of biomass containing 60% of P3HP (i.e. 1.2 g of P3HP) is extracted with 20 g of dimethyl sulfone at 115° C. for 2 h.

The insoluble organic waste is separated by centrifugation and weighs 0.8 g. A maximum of 1.2 g of P3HP has therefore been extracted into the dimethyl sulfone, i.e. potentially all of the P3HP contained in the biomass.

The homogeneous P3HP-dimethyl sulfone mixture is introduced directly into the thermolysis assembly. 0.02 g of HQME is added as polymerization inhibitor. The thermolysis reaction is carried out at 200° C. and 20 kPa for 4 h. 1.1 g of AA having a purity greater than 90% is recovered in the overhead receiver. The residue in the thermolysis reactor is a clear liquid consisting essentially of dimethyl sulfone which can be recycled to the extraction step.

### Experiment 11

2 g of biomass containing 60% of P3HP (i.e. 1.2 g of P3HP) is extracted with 20 g of propylene carbonate at 100° C. for 2 h.

The insoluble organic waste is separated by centrifugation and weighs 0.8 g. A maximum of 1.2 g of P3HP has therefore been extracted into the propylene carbonate, i.e. potentially all of the P3HP contained in the biomass.

The homogeneous P3HP-propylene carbonate mixture is introduced directly into the thermolysis assembly. 0.02 g of HQME is added as polymerization inhibitor. The thermolysis reaction is carried out at 200° C. and 20 kPa for 4 h. 1.0 g of AA having a purity greater than 90% is recovered in the overhead receiver. The residue in the thermolysis reactor is a clear liquid consisting essentially of propylene carbonate which can be recycled to the extraction step.

1. A process for producing α,β-unsaturated carboxylic acids from biomass containing poly(3-hydroxyalkanoate) (P3HA), said process comprising the steps of:
extracting P3HA from the biomass with a solvent capable of solubilizing P3HA to form a P3HA-solvent mixture,
separating organic waste insoluble in said solvent from the P3HA-solvent mixture,
thermolyzing said P3HA-solvent mixture to obtain said α,β-unsaturated carboxylic acid and recover said solvent.

2. The process as claimed in claim 1, wherein the biomass is pretreated by means of washing, drying or milling operations until the biomass contains at least 30% by weight of P3HA.

3. The process as claimed in claim 1, wherein the extracting is carried out at a temperature of between 20° C. and 130° C.

4. The process as claimed in claim 1, wherein the thermolyzing is carried out at a temperature of between 130° C. and 300° C., and at a pressure of between 1 kPa and 101 kPa (atmospheric pressure).

5. The process as claimed in claim 1, wherein the solvent has a boiling temperature at atmospheric pressure of greater than 230° C.

6. The process as claimed in claim 1, wherein solubility of the P3HA in the solvent in the extracting step is greater than 5% by weight.

7. The process as claimed in claim 1, wherein said extracting, separating and thermolyzing are each performed batchwise or continuously.

8. The process as claimed in claim 1, wherein said thermolyzing takes place in the presence of one or more polymerization inhibitors.

9. The process as claimed in claim 8, wherein an amount of the one or more polymerization inhibitors ranges from 0.005% to 5% by weight relative to a weight of the poly(3-hydroxyalkanoate).

10. The process as claimed in claim 8, wherein the one or more polymerization inhibitors are selected from phenol derivatives, phenothiazine derivatives, nitroxide derivatives or para-phenylenediamine derivatives.

11. The process as claimed in claim 8, wherein said one or more polymerization inhibitors is hydroquinone methyl ether (HQME).

12. The process as claimed in claim 1, wherein the solvent is selected from glycol diethers (glymes), sulfur-containing organic solvents, or organic carbonates.

13. The process as claimed in claim 8, wherein the solvent is itself a polymerization inhibitor.

14. The process as claimed in claim 1, further comprising recycling the solvent recovered from the thermolyzing step to the extracting step.

15. The process as claimed in claim 1, wherein the poly(3-hydroxyalkanoate) contains a 3-hydroxypropionate unit and at least one of the α,β-unsaturated carboxylic acids produced is acrylic acid.

16. The process as claimed in claim 1, wherein the poly(3-hydroxyalkanoate) is poly(3-hydroxypropionate) and the α,β-unsaturated carboxylic acid produced is acrylic acid.

17. The process as claimed in claim 1, wherein the poly(3-hydroxyalkanoate) contains a 3-hydroxybutyrate unit and at least one of the α,β-unsaturated carboxylic acids produced is crotonic acid.

18. The process as claimed in claim 1, wherein the poly(3-hydroxyalkanoate) is poly(3-hydroxybutyrate) and the α,β-unsaturated carboxylic acid produced is crotonic acid.

19. The process as claimed in claim 1, wherein the poly(3-hydroxyalkanoate) contains a 3-hydroxyisobutyrate unit and at least one of the α,β-unsaturated carboxylic acids produced is methacrylic acid.

20. The process as claimed in claim 1, wherein the poly(3-hydroxyalkanoate) is poly(3-hydroxyisobutyrate) and the α,β-unsaturated carboxylic acid produced is methacrylic acid.

21. A process for producing α,β-unsaturated carboxylic acids according to claim 1, further comprising a step of condensing the produced α,β-unsaturated carboxylic acids, followed by one or more purification steps selected from distillation, liquid/liquid extraction, separation using a film evaporator, or crystallization, or a combination of these techniques.
